(19) United States
(12) Patent Application Publication
(10) Pub. No.: US 2010/0087523 A1
(43) Pub. Date: Apr. 8, 2010

(54) Rupestonic acid derivatives and use thereof

(75) Inventors: Haji Akber Aisa, Xinjiang (CN); Jianping Yong, Xinjiang (CN); Jiangyu Zhao, Xinjiang (CN)

Correspondence Address: ROBERT E. BUSHNELL & LAW FIRM, 2029 K STREET NW, SUITE 600, WASHINGTON, DC, 20006-1004, US

(73) Assignee: Xinjiang Technical Institute of Physics & Chemistry, Chinese Academy of Sciences, Xinjiang (CN)

(21) Appl. No.: 12/585,447

(22) Filed: Sep. 15, 2009

(30) Foreign Application Priority Data
Oct. 8, 2008 (CN) 200810072964.1

(51) Int. Cl. A61K 31/215; C07C 237/18; C07C 59/48; A61K 31/16; A61P 31/12; A61P 31/22

(52) U.S. Cl. 514/530; 564/189; 562/462; 514/613

(57) ABSTRACT

Rupestonic acid derivatives are rupestonic acid derivative A- or B-type compounds, prepared through a condensation reaction from starting materials, i.e., a monomer compound of rupestonic acid and an aromatic amine or fatty amine or an organic alcohol, i.e., a fatty alcohol or an aromatic alcohol. Rupestonic acid, which is the monomer compound of sesquiterpene isolated from the plant Xinjiang Artemisia rupestric L., is used as a mother compound. Natural anti-virus lead compound with high activity are discovered by modifying the structure of the monomer compound of rupestonic acid. Activity results of the lead compound showed pharmaceutical application of anti-I, II flu virus and anti-I, II herpes simplex virus infection. These compounds can be used alone or in combination with one or more pharmaceutical acceptable, inert and nontoxic excipients or carries in a pharmaceutical composition.

### CLAIM OF PRIORITY

This application makes reference to, incorporates the same herein, and claims all benefits accruing under 35 U.S.C. §119 from an application earlier filed in the State Intellectual Property Office of the People's Republic of China on 8 Oct. 2008 and there duly assigned Serial No. 200810072964.1.

### FIELD OF THE INVENTION

The present invention relates to rupestonic acid derivatives and the use thereof. In particular, such derivatives have a structure that provides anti-flu virus and anti-herpes virus properties. The present invention also relates to pharmaceutical compositions containing the same.

### BACKGROUND OF THE INVENTION

Humanity has been fighting viral infections for many years. Based on an analysis of the degree of undercalculation, 60% of epidemic infectious diseases were caused by viral infections; however, there is no efficient and specialized antiviral drug until now. Abuse of antibiotics and antiviral drugs have brought an increase in resistance and variability of viruses, which has led to the appearance of new viruses. Therefore, there is an urgent need to develop antiviral drugs with novel structures and functional mechanisms to satisfy social and clinical needs.

Drugs obtained purely from chemosynthesis have a high activity, strong side effects, and a high elimination rate during clinical trials. Particularly, the development of anti-virul drugs is restricted by many factors and much more difficult because of its higher clinical elimination rates. According to a calculation, the elimination rate of anti-viral drugs entering Phase I-III trials reaches 70% or more. Therefore, seeking a lead compound with a novel structure from natural products, further modifying the existing structures, and synthesizing a “me-too” drug, have become standard approaches for developing novel medicines.

The Feverfew, Xinjiang Artemisia rupestric L., has long been used in folk remedies of Xinjiang, China. It possesses properties such as anti-inflammation, anti-allergy, anti-tumor, immune-boosting, anti-bacteria, detoxification, liver-protection, and the like. Compound medicines containing Xinjiang Artemisia rupestric L. as the main component have been used clinically. For example, the compound Artemisia rupestric L. granule, developed by Xinjiang Xiyu Pharmaceutical Co., Ltd., is clinically used to treat diseases such as virus, cold, sore throat, etc. Among others, rupestonic acid is a monomer sesquiterpene with multi functional groups, which is isolated from Xinjiang Artemisia rupestric L.

At earlier investigative stages of the present invention, a series of rupestonic acid derivatives have been synthesized. All the derivatives were screened for in vitro anti-flu virus and anti-herpes simplex virus activities. The result of the activity screen showed that most compounds have higher anti-flu virus activity and some of the derivatives have higher anti-herpes simplex virus activity. The present invention is the result of further investigations of the work disclosed in CN2007101800216. The structure of rupestonic acid is further derived, so as to find a highly activated anti-virul lead compound with natural products as its mother compound.

In the present invention, most of the compounds possess higher inhibition against A-type flu virus, and the anti-flu viral activity of some compounds is higher than that of the compounds in patent CN2007101800216. For example, compounds B-2 and B-9 inhibit A-type flu virus, with an IC₅₀ of 19.1 μM and 13.4 μM, respectively, and a higher selectivity index of 12.6 and 14.3, respectively.

### BRIEF SUMMARY OF INVENTION

One object of the invention to provide improved rupestonic acid derivatives and improved use thereof.

Another object is to provide improved pharmaceutical compositions containing rupestonic acid derivatives and improved use thereof.

Still another object is to synthesize rupestonic acid derivatives with the structure shown in Scheme I as formulas A and B, and pharmaceutically acceptable salts thereof. The compounds of the invention increase the pharmacological activity of rupestonic acid while decreasing side effects.

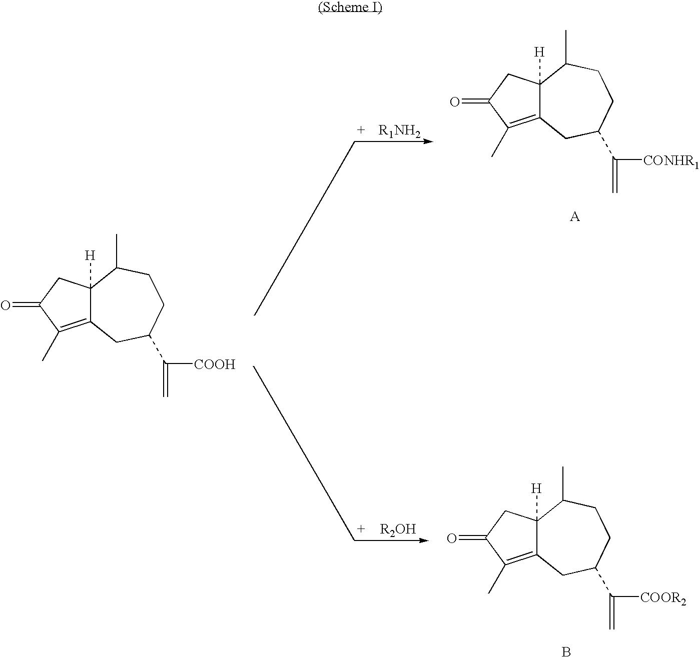

In the rupestonic acid derivative A-type compound (formula A), R₁ is n-dodecyl, p-chlorophenyl, o-chlorophenyl, p-bromophenyl, o-bromo-phenyl, 2,4-dichlorophenyl or benzyl.

In the rupestonic acid derivative B-type compound (formula B), R₂ is ethyl or p-chlorophenyl, o-chlorophenyl, p-bromophenyl, o-bromophenyl, p-fluorophenyl, 2,4-dichlorophenyl, p-hydroxyphenyl, p-methylphenyl, benzyl, p-fluorobenzyl, p-trifluoromethylbenzyl, p-chlorobenzyl, p-nitrylbenzyl, p-bromobenzyl, o-bromobenzyl, p-methoxybenzyl, 2,4-dichlorobenzyl, or o-chlorobenzyl.

Another object of the invention is to provide a pharmaceutical composition containing rupestonic acid derivatives with formulas A and B or their derivatives with one or more pharmaceutically acceptable inertial carriers, excipients or diluents. The present compositions have, for example, anti-flu virus and anti-herpes virus properties. The compositions can thus be used in a method of treating flu-virus or herpes virus by administering to a patient in need thereof a pharmaceutically effective amount of a composition comprises a compound of formula A or formula B.

### DETAILED DESCRIPTION OF INVENTION

The invention provides rupestonic acid derivatives. The rupestonic acid derivatives are a rupestonic acid derivative-type compound as shown in the formulas A and B of scheme I or pharmaceutically acceptable salts thereof. The rupestonic acid derivative compounds are a rupestonic acid derivative A- or B-type compound. A rupestonic acid derivative A-type compound (formula A) is prepared from a rupestonic acid monomer compound and an amine compound through an acylamide reaction. A rupestonic acid derivative B-type compound (formula B) is prepared from a rupestonic acid monomer compound and an alcohol compound through an esterification reaction. The amine compound is a fatty amine or an aromatic amine; the alcohol compound is a fatty alcohol or an aromatic alcohol.

Scheme I is as follows:

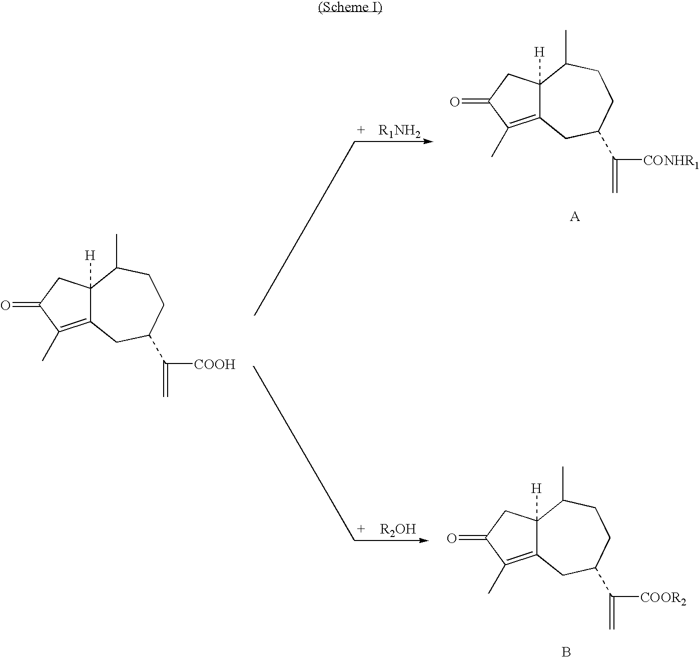

In the rupestonic acid derivative A-type compound (formula A), R₁ is n-dodecyl, p-chlorophenyl, o-chlorophenyl, p-bromophenyl, o-bromo-phenyl, 2,4-dichlorophenyl or benzyl.

In the rupestonic acid derivative B-type compound (formula B), R₂ is ethyl or p-chlorophenyl, o-chlorophenyl, p-bromophenyl, o-bromophenyl, p-fluorophenyl, 2,4-dichlorophenyl, p-hydroxlyphenyl, p-methylphenyl, benzyl, p-fluorobenzyl, p-trifluoromethylbenzyl, p-chlorobenzyl, p-nitrylbenzyl, p-bromobenzyl, o-bromobenzyl, p-methoxybenzyl, 2,4-dichlorobenzyl, or o-chlorobenzyl.

A rupestonic acid derivative A-type compound (formula A) is preferably selected from any one of the following compounds:
nitrogen (n-dodecyl)-Rupestonic amide,
nitrogen (4-chloro-phenyl)-Rupestonic amide,
nitrogen (2-chloro-phenyl)-Rupestonic amide,
nitrogen (4-bromo-phenyl)-Rupestonic amide,
nitrogen (2-bromo-phenyl)-Rupestonic amide,
nitrogen (2,4-dichloro-phenyl)-Rupestonic amide, or
nitrogen (benzyl)-Rupestonic amide.

A rupestonic acid derivative B-type compound (formula B) is preferably selected from any one of the following compounds:
Ethyl Rupestonic ester,
Rupestonic (p-chlorophenyl)-ester,
Rupestonic (o-chlorophenyl)-ester,
Rupestonic (p-bromophenyl)-ester,
Rupestonic (o-bromophenyl)-ester,
Rupestonic (p-fluorophenyl)-ester,
Rupestonic (2,4-dichloro-phenyl)-ester,
Rupestonic (p-hydroxyl-phenyl)-ester,
Rupestonic (p-methylphenyl)-ester,
Rupestonic (p-cyano-phenyl)-ester,
Benzyl Rupestonic ester,
Rupestonic (p-fluorobenzyl)-ester,
Rupestonic (p-trifluoromethyl-benzyl)-ester,
Rupestonic (p-chlorobenzyl)-ester,
Rupestonic (p-nitryl-benzyl)-ester,
Rupestonic (p-bromobenzyl)-ester,
Rupestonic (o-bromobenzyl)-ester,
Rupestonic (p-methoxy-benzyl)-ester,
Rupestonic (2,4-dichloro-benzyl)-ester,
Rupestonic (o-chlorobenzyl)-ester, or
Rupestonic (p-cyano-benzyl)-ester.

The pharmaceutically acceptable salts are formed from a rupestonic acid derivative A-type compound and a pharmaceutically acceptable acid. Inorganic salts are, for example, hydrochloric salts, sulphate, phosphate, or nitrate. Organic salts are, for example, formate, oxalate, acetate, citrate, fumarate, maleate, or amino acid salts.

The compounds, as an active ingredient, can form pharmaceutical compositions either alone or in combination with one or more pharmaceutically acceptable inert and nontoxic excipients or carriers.

The rupestonic acid derivatives are used, for example, to prepare medicines for treating anti-A, B-type flu viruses.

The rupestonic acid derivatives are used to prepare, for example, medicines for treating anti-I, II-type herpes simplex virus infections.

A compound with a formula A or B described above may be synthesized by the following methods.

### Synthesis of a Rupestonic Acid Derivative A-Type Compound

In a THF system, a rupestonic acid derivative A-type compound (formula A) is synthesized from starting materials, e.g., a rupestonic acid and an aromatic amine or a fatty amine shown in Scheme II, under the action of a condensation agent, e.g., DCC/HOBt. R₁ is n-dodecyl, p-chlorophenyl, o-chlorophenyl, p-bromophenyl, o-bromophenyl, 2,4-dichlorophenyl or benzyl.

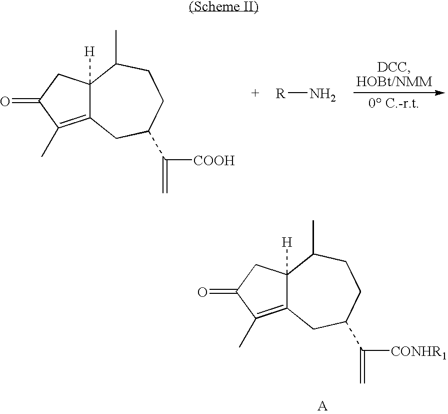

### Synthesis of a Rupestonic Acid Derivative B-Type Compound (Formula B)

In a THF/acetonitrile system (Scheme III), a rupestonic acid derivative B-type compound is synthesized from starting materials, e.g., a rupestonic acid and an organic alcohol such as a fatty alcohol or aromatic alcohol under the action of a condensation agent e.g., DCC/DMAP. R₂ is ethyl or p-chlorophenyl, o-chlorophenyl, p-bromophenyl, o-bromophenyl, p-fluorophenyl, 2,4-dichlorophenyl, p-hydroxylphenyl, p-methylphenyl, benzyl, p-fluorobenzyl, p-trifluoromethylbenzyl, p-chlorobenzyl, p-nitrylbenzyl, p-bromobenzyl, o-bromobenzyl, p-methoxybenzyl, 2,4-dichlorobenzyl, or o-chlorobenzyl.

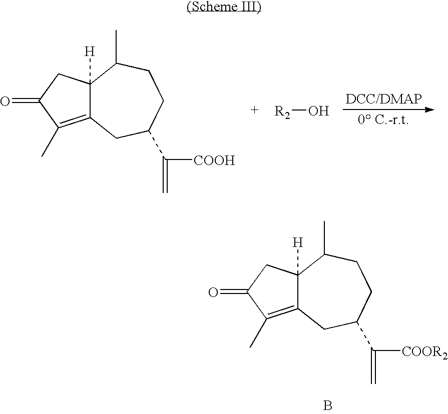

As active ingredients, the salts of the compounds shown by formula (B) shown above or the salts of the rupestonic acid derivative A-type compounds, can be used to prepare medicines for treating, for example, anti-flu virus and anti-herpes virus.

The compounds of formulas A or B of Scheme I, and pharmaceutically acceptable salts thereof, can be combined with one or more pharmaceutically acceptable carriers, excipients or diluents, thereby forming a pharmaceutical composition. According to conventional pharmaceutics, such pharmaceutical compositions can be manufactured into various pharmaceutical formulations, e.g., solid oral agent, liquid oral agent or liquid injection, etc.

The bioactivities of the compounds of the formulas A or B of scheme I can be determined by the following methods.

### 1. Measurement of Anti-A, B-Type Flu Virus Activities

MDCK (Madin darby canine kidney) cells are used as host cells for viruses. For each sample, the inhibition of virus-induced cytopathic effect (CPE) is measured.

### Testing Materials and Method:

(1) Virus sp.: in September, 2006, A-type flu virus (jifang/90-15) and B-type flu virus (jifang/97-13) were cultured in allantoic cavities of chick embryos for passage, and the culture was stored at −80° C.

(2) Treatment of samples: samples are dissolved in DMSO, brought to a suitable initial concentration by adding the culture fluid, and diluted three (3) times with the culture fluid, with a dilution factor of eight (8) for each.

(3) Positive control: ribovirin (RBV), manufactured by Zhejiang Kangyu Pharmaceutical Co., Ltd. (batch number 960501).

(4) Testing method: MDCK cells are inoculated in a 96-well culture plate, cultured in 5% CO₂, 37° C. for 24 hours. A-type flu viruses 10⁻³ (100×TCID₅₀) and B-type flu viruses 10⁻² (30×TCID₅₀) are added into the MDCK cells. After adsorption at 37° C. for 2 hours, the virus solution is decanted, and the drugs with various dilution factors are added respectively. The virus control and cell control are set up, cultured at 37° C. for 36 hours, and observed. CPE is recorded and anti-flu virus inhibition concentration 50% (IC₅₀) is calculated for each sample.

### 2. Measurement of Anti-I, H Herpes Simplex Virus Activities

Vero cells are used as host cells for viruses. For each sample, the inhibition of I, II herpes virus-induced cytopathic effect (CPE) is measured.

### Testing Materials and Method

(1) Virus sp.: HSV-I, VR733 sp., HSV-II, SAV sp., both provided by ATCC.

(2) Treatment of samples: All reagents should be freshly prepared prior to use. samples are dissolved in DMSO, brought to a suitable concentration, and diluted 3 times with the culture fluid during the examination, with a dilution factor of 8 for each.

(3) Positive control: acyclovir (ACV), manufactured by Hubei Keyi Pharmaceutical, Co.

(4) Testing method: Vero cells are inoculated in a 96-well culture plate. The cells were grown at 37° C. in a humidified 5% CO₂ for 24 hours. I-type herpes viruses 10⁻³ (50×TCID₅₀ infection amount) and II-type herpes viruses 10⁻⁴ (17×TCID₅₀ infection amount) are added into Vero cells respectively. After adsorption for 2 hours, the medium is removed, and the samples and the positive control are added according to the above dilution factors. At the same time, a cell control well and a virus control well are set up. After the observation of cell pathological extent (CPE) for 48 hours, the inhibition concentration 50% (IC₅₀) of 1-type and II-type herpes simplex viruses is calculated for each sample.

The present invention is further illustrated by the following examples. Although the Examples are given to further illustrate the invention, however, the invention is not limited to the included Examples.

### Equipment and Reagents:

Varian 600 Nuclear Magnetic resonance instrument (CDCl₃,TMS as an internal reference), HP1100 LC/MS. D, Shimadzu FTIR-8400S (produced by Shimadzu Company, Japan), all melting points were determined on Yanaco MP-300 micro melting point apparatus and values are uncorrected.

Rupestonic acid was isolated by conventional methods, with a purity of 98% tested by HPLC. Other reagents are all commercial and analytically pure.

### Example 1

### Preparation of Rupestonic Acid

Rupestonic acid was extracted by a conventional continuous extraction method. Five kilograms of ground Artemisia rupestric L. were used. The extraction was carried out with IL 95% Ethanol three (3) times, and the resultant extracts were combined. The combined extracts were concentrated into an extractum, which was further extracted with ethyl acetate several times. The ethyl acetate layers were combined, concentrated and separated through a silica gel column. The resultant raw rupestonic acid was re-crystallized to obtain the pure product.

Rupestonic acid: colorless column crystal, [a]_{D}²⁶+150 (c 0.176, CH₃CH₂OH), mp.132-134° C. IR (KBr) υ: 3230, 2970-2860, 1720, 1680, 1635, 1415, 1390, 1238, 958 cm⁻¹;

¹H NMR (600 MHz, CDCl₃): 0.67 (d, J=7.2 Hz, 3H,CH₃), 1.63 (m, 1H), 1.64 (m, 1H), 1.81 (m, 1H), 1.84 (m, 1H), 1.88 (m, 1H), 2.06 (m, 1H), 2.14 (m, 1H), 2.46 (m, 1H), 2.64 (m, 1H), 2.86 (m, 1H), 2.90 (m, 1H), 3.22 (m, 1H), 5.76 (s, 1H), 6.40 (s, 1H); ¹³CNMR (150 MHz, CDCl₃): 7.9, 12.0, 31.4, 35.1, 36.4, 37.5, 38.2, 41.1, 45.9, 125.2, 137.6, 145.6, 171.3, 175.2, 208.8;

ESI-MS (m/z): 519[2M+23]⁺, 497[2M+1]^{±}, 249[M+1]⁺.

The absolute configuration of rupestonic acid was determined by XRD monocrystal diffraction.

### Preparation of A-Type Rupestonic Acid Derivatives

The preparation of A-type rupestonic acid derivatives, for example, through the synthesis of N-(2,4-dichloro-phenyl)-Rupestonic amide A-6 may be achieved in the following process steps:

Eight milliliters of dry THF was added into a 25 mL round-bottom flask containing 0.124 g (0.5 mmol) rupestonic acid and 0.113 g (0.55 mmol) DCC. The mixture was reacted for 10 minutes with ice-bath while being stirred. Subsequently, while the stirring and ice-bath conditions are maintained, 0.08 g (0.6 mmol) HOBt dissolved in 2 milli-Liters of dry THF was added into the reaction mixture by injection. After the resultant mixture was reacted for 30 minutes, 0.55 mmol p-2,4-dichloro-benzyl amine dissolved in 3 milli-Liters dry THF, was dripped into the reaction system and reacted for 30 minutes in an ice-bath. The reaction temperature was raised naturally to room temperature (RT). The completion of the reaction was determined by TLC. Then the reaction solution was vacuum-concentrated, and the target product compound A-6 was obtained by directly separating the concentrated residues through a column (V(petroleum ether):V(ethyl acetate)=5:1-2:1). Other A-type compounds were synthesized in accordance with the experimental procedure for compound A-6. The structures of all A-type compounds were characterized by various analysis methods, such as IR, ¹H NMR, ESI-MS, etc., and the results are shown in the tables below.

TABLE 1
Structures and names of A-type compounds

| Formula | R₁ | Code | Name |
|---|---|---|---|
| 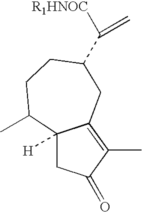 | n-dodecyl 4-chloro-phenyl 2-chloro-phenyl 4-bromo-phenyl 2-bromo-phenyl 2,4-dichloro-phenyl benzyl | A-1 A-2 A-3 A-4 A-5 A-6 A-7 | N(n-dodecyl)-Rupestonic amide N(4-chloro-phenyl)-Rupestonic amide N(2-chloro-phenyl)-Rupestonic amide N(4-bromo-phenyl)-Rupestonic amide N(2-bromo-phenyl)-Rupestonic amide N(2,4-dichloro-phenyl)-Rupestonic amide N(benzyl)-Rupestonic amide |

TABLE 2
Physical constants, as well as IR and MS data of A-type compounds

| | | melting | | |
|---|---|---|---|---|
| code | physical state | point/° C. | IR/cm⁻¹ | MS(%) |
| A-1 | White solid | 79-80 | 3283, 2961, 2916, 1693, 1657, 1628, 1593, | 416([M + 1]⁺, 35), |
| | | | 1514, 1317. | 438([M + 23]⁺, 18). |
| A-2 | White and | 57-58 | 3300, 2951, 2916, 1693, 1658, 1627, 1595, | 358([M + 1]⁺, 80), |
| | foam-like solid | | 1537, 1440, 1321, 825. | 380([M + 23]⁺, 90). |
| A-3 | Light yellow | | 3320, 2951, 2916, 1693, 1658, 1627, 1595, | 358.6([M + 1]⁺, 80). |
| | and wax-like | | 1537, 1440, 1321, 835. | |
| A-4 | Colorless and | 62-63 | 3320, 2951, 2916, 1693, 1658, 1627, 1595, | 425([M + 23]⁺, 76). |
| | foam-like solid | | 1537, 1440, 1321, 835. | |
| A-5 | Colorless and | 118-119 | 3320, 2951, 2916, 1693, 1658, 1627, 1595, | 403.1([M+1]⁺, 80). |
| | foam-like solid | | 1537, 1440, 1321, 835. | |
| A-6 | Light yellow | | 3320, 2951, 2916, 1693, 1658, 1627, 1595, | 393.1([M + 1]⁺, 80) |
| | oil | | 1537, 1440, 1321, 835. | |
| A-7 | White solid | 118-119 | 3300, 2951, 2916, 1693, 1658, 1627, 1595, | 338([M + 1]+, 96), |
| | | | 1537, 1440, 1321, 759 | 360([M + 23]+, 88) |

TABLE 3
¹H NMR data of A-type compounds

| code | ¹H NMR(CDCl₃) |
|---|---|
| A-1 | 0.64-0.66 (d, 3H, CH₃), 0.86-0.90 (m, 3H, CH₃), 1.21-1.32 (m, 18H, 9CH₂), 1.51-1.57 (m, 4H), 1.65 |
| | (s, 3H, CH₃), 1.78-1.79 (m, 1H), 1.81-1.84 (m, 1H), 2.01-2.06 (d, 1H), 2.13 (m, 1H), 2.43-2.46 (m, |
| | 1H), 2.48-2.50 (m, 1H), 2.85-2.96 (m, 2H), 3.18 (m, 1H), 3.29-3.34 (m, 2H, Ph-CH₂), 5.32 (s, 1H), |
| | 5.46 (s, 1H), 5.83 (brs, 1H, NH—C═O). |
| A-2 | 0.66 (d, J = 7.2 Hz, 3H, CH₃), 1.67-1.69 (s, 4H, 2CH₂), 1.87 (s, 3H, CH₃), 2.03 (m, 1H), 2.14 (m, |
| | 1H), 2.52-2.62 (m, 2H, CH₂), 2.91-2.95 (m, 1H), 3.01-3.05 (m, 1H), 3.20 (m, 1H), 5.60 (s, 1H), |
| | 6.13 (s, 1H), 6.51 (brs, 1H, CONH—), 7.01-7.05(m, 2H), 7.50-7.54(m, 2H). |
| A-3 | 0.66 (d, J = 7.2 Hz, 3H, CH₃), 1.67-1.69 (s, 4H, 2CH₂), 1.87 (s, 3H, CH₃), 2.03 (m, 1H), 2.14 (m, |
| | 1H), 2.52-2.62 (m, 2H, CH₂), 2.91-2.95 (m, 1H), 3.01-3.05 (m, 1H), 3.20 (m, 1H), 5.56 (s, 1H), |
| | 6.18 (s, 1H), 6.55(brs, 1H, CONH—), 7.19-7.26(m, 2H), 7.30-7.34(m, 1H), 7.46-7.48(d, d, 1H). |
| A-4 | 0.66 (d, J = 7.2 Hz, 3H, CH₃), 1.67-1.69 (s, 4H, 2CH₂), 1.87 (s, 3H, CH₃), 2.03 (m, 1H), 2.14 (m, |
| | 1H), 2.52-2.62 (m, 2H, CH₂), 2.91-2.95 (m, 1H), 3.01-3.05 (m, 1H), 3.20 (m, 1H), 5.60 (s, 1H), |
| | 6.13 (s, 1H), 6.51 (brs, 1H, CONH—), 7.01-7.05 (m, 2H), 7.50-7.54 (m, 2H). |
| A-5 | 0.66 (d, J = 7.2 Hz, 3H, CH₃), 1.65-1.71 (s, 4H, 2CH₂), 1.87 (s, 3H, CH₃), 2.05 (m, 1H), 2.16 (m, |
| | 1H), 2.52-2.62 (m, 2H, CH₂), 2.91-2.95 (m, 1H), 3.01-3.05 (m, 1H), 3.20 (m, 1H), 5.66 (s, 1H), |
| | 6.13 (s, 1H), 6.53 (brs, 1H, CONH—), 7.17-7.28 (m, 2H), 7.31-7.35 (m, 1H), 7.48-7.50 (d, d, 1H). |
| A-6 | 0.66 (d, J = 7.2 Hz, 3H, CH₃), 1.65-1.71 (s, 4H, 2CH₂), 1.87 (s, 3H, CH₃), 2.05 (m, 1H), 2.16 (m, |
| | 1H), 2.52-2.62 (m, 2H, CH₂), 2.91-2.95 (m, 1H), 3.01-3.05 (m, 1H), 3.20 (m, 1H), 5.65 (s, 1H), |
| | 6.12 (s, 1H), 6.55 (brs, 1H, CONH—), 7.14-7.16 (d, J = 8.0 Hz, 1H), 7.28-7.31(m, 1H), 7.48 (d, 1H). |
| A-7 | 0.66 (d, J = 7.2 Hz, 3H, CH3), 1.67-1.69 (brs, 4H, 2CH2), 1.87 (s, 3H, CH3), 2.03-2.04 (m, |
| | 1H), 2.14-2.15 (m, 1H), 2.52-2.62 (m, 2H, CH2), 2.91-2.95 (m, 1H), 3.01-3.05 (m, |
| | 1H), 3.20-3.21 (m, 1H), 4.45-4.48 (m, 2H, Ph-CH2), 5.48 (s, 1H), 5.69 (s, 1H), |
| | 6.18-6.20 (brs, 1H, NH—C═O), 7.28-7.38 (m, 5H, Ph-H) |

### Example 2

Rupestonic acid was prepared according to Example 1.

Synthesis of B-type rupestonic acid derivatives (for example, the synthesis of rupestonic acid (p-methyl-phenyl)-ester)

Eight milliliters of dry THF was added into a 25 mL round-bottom flask containing 0.124 g (0.5 mmol) rupestonic acid and 0.113 grams (0.55 mmol) DCC. While being stirred, 0.031 grams (0.25 mmol) DMAP was added into the reaction system in an ice-bath. After 30 minutes of reaction, 0.128 g (0.60 mmol) p-methyl phenol was added into the reaction system and the reaction was carried out in an ice-bath for another 30 minutes while being stirred. The reaction temperature was then raised naturally to RT (i.e., room temperature) and maintained at RT for 8 hours. Subsequently, DCU i.e., the precipitate, was filtered. The remaining filtrate was directly separated through a silicon column after being concentrated, and the target product compound B-9 was obtained. Other B-type compounds were synthesized in accordance with the experimental procedure for compound B-9. All of the synthesized B-type compounds were characterized by various analysis methods, such as IR, ¹H NMR, ESI-MS, etc., and a part of the ¹H NMR data is shown below.

TABLE 4
Structures and names of B-type compounds

| formula | R₂ | code | Name |
|---|---|---|---|
| 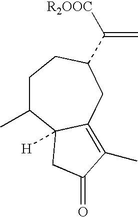 | ethyl p-chlorophenyl o-chlorophenyl p-bromophenyl o-bromophenyl p-fluorophenyl 2,4-dichloro-phenyl p-hydroxyl-phenyl p-methyl phenyl p-cyano-phenyl benzyl p-fluorobenzyl p-trifluoromethyl-benzyl p-chlorobenzyl 4-nitryl-benzyl | B-1 B-2 B-3 B-4 B-5 B-6 B-7 B-8 B-9 B-10 B-11 B-12 B-13 B-14 B-15 | Ethyl Rupestonic ester Rupestonic (p-chlorophenyl)-ester Rupestonic (o-chlorophenyl)-ester Rupestonic (p-bromophenyl)-ester Rupestonic (o-bromophenyl)-ester Rupestonic (p-fluorophenyl)-ester Rupestonic (2,4-dichloro-phenyl)-ester Rupestonic (p-hydroxyl-phenyl)-ester Rupestonic (p-methylphenyl)-ester Rupestonic (p-cyano-phenyl)-ester Benzyl Rupestonic ester Rupestonic (p-fluorobenzyl)-ester, Rupestonic (p-trifluoromethyl-benzyl)-ester Rupestonic (p-chlorobenzyl)-ester Rupestonic (p-bromobenzyl)-ester |
| | p-bromobenzyl | B-16 | Rupestonic (4-chloro-benzyl)-ester |
| | o-bromobenzyl | B-17 | Rupestonic (o-bromobenzyl)-ester |
| | p-methoxyl-benzyl | B-18 | Rupestonic (p-methoxy-benzyl)-ester |
| | 2,4-dichloro-benzyl | B-19 | Rupestonic (2,4-dichloro-benzyl)-ester |
| | o-chlorobenzyl | B-20 | Rupestonic (o-chlorobenzyl)-ester |
| | p-cyano-benzyl | B-21 | Rupestonic (p-cyano-benzyl)-ester |

TABLE 5
Physical constants, as well as IR and MS data of A-type compounds

| | | melting | | |
|---|---|---|---|---|
| code | physical state | point/° C. | IR/cm⁻¹ | MS(%) |
| B-1 | yellow oil | | 3032, 2951, 2924, 1712, 1662, 1566, | 277([M + 1]⁺, 96), |
| | | | 1450, 1375, 1339, 1240, 1145. | 299([M + 23]⁺, 100), |
| | | | | 315([M + 39]⁺, 10). |
| B-2 | Colorless solid | 91-93 | 2959-2878, 1713, 1694, 1627, 1512, | |
| | | | 1223, 1141, 826, 748. | |
| B-3 | Light yellow oil | | 2959-2878, 1713, 1694, 1627, 1512, | |
| | | | 1223, 1141, 826, 748. | |
| B-4 | colorless oil | | 2959-2878, 1713, 1694, 1627, 1512, | |
| | | | 1223, 1141, 826, 748. | |
| B-5 | colorless oil | | 2959-2878, 1713, 1694, 1627, 1512, | |
| | | | 1223, 1141, 826, 748. | |
| B-6 | Colorless solid | 89-90 | 2959-2878, 1713, 1694, 1627, 1512, | |
| | | | 1223, 1141, 826, 748. | |
| B-7 | Light yellow oil | | 2959-2878, 1713, 1694, 1627, 1512, | |
| | | | 1223, 1141, 826, 748. | |
| B-8 | White solid | 119-121 | 3032,2951,2924, 1712, 1662, 1618, | 339([M − 1]⁻, 45), |
| | | | 1566, 1450, 1375, 1339, 1240, 1145. | 240([M]⁺, 8 ). |
| B-9 | White solid | 110-111 | 3032, 2951, 2924, 1712, 1662, 1618, | 339([M + 1]⁺, 60), |
| | | | 1566, 1450, 1375, 1339, 1240, 1145, 918. | 361([M + 23]⁺, 100). |
| B-11 | colorless oil | | 2959-2878, 1713, 1694, 1627, 1512, | 339 ([M + 1]⁺, 65). |
| | | | 1223, 1141, 826. | |
| B-12 | colorless oil | | 2959-2878, 1713, 1694, 1627, 1512, | 356([M]⁺, 100), |
| | | | 1223, 1141, 826, 748. | 379([M + 23]+, 95) |
| B-13 | colorless oil | | 2959-2878, 1713, 1694, 1627, 1512, | 407([M + 1]⁺, 100), |
| | | | 1223, 1141, 826, 748. | 429([M + 23]⁺, 10). |
| B-14 | colorless oil | | 2959-2878, 1713, 1694, 1627, 1512, | 373([M]⁺, 100), |
| | | | 1223, 1141, 826, 748. | 374([M + 1]⁺, 45), |
| | | | | 391([M + 18]⁺, 15). |
| B-15 | Light yellow oil | | 2959-2878, 1713, 1694, 1627, 1512, | 384([M + 1]⁺, 70), |
| | | | 1223, 1141,826, 748. | 401([M + 18]⁺, 30), |
| | | | | 406([M + 23]⁺, 10). |
| B-16 | colorless and oily | | 2959-2878, 1713, 1694, 1627, 1512, | 418([M + 1]⁺, 50) |
| | | | 1223, 1141, 826, 748. | |
| B-17 | Ligjt yellow and | | 2959-2878, 1713, 1694, 1627, 1512, | 417([M]⁺, 15), |
| | oily | | 1223, 1141, 826, 748. | 435([M + 18]⁺, 60). |
| B-18 | Ligjt yellow and | | 2959-2878, 1713, 1694, 1627, 1512, | 369 ([M + 1]⁺, 40). |
| | oily | | 1223, 1141, 826, 748. | |
| B-19 | colorless and oily | | 2959-2878, 1713, 1694, 1627, 1512, | 407([M] ⁺, 56) |
| | | | 1223, 1141, 826, 748. | |
| B-20 | colorless and oily | | 2959-2878, 1713, 1694, 1627, 1512, | 391([M + 18]⁺, 80). |
| | | | 1223, 1141, 826, 748. | |

TABLE 5
Part of the ¹H NMR data of B-type compounds

| code | ¹H NMR(CDCl₃) |
|---|---|
| B-1 | 0.66 (d, J = 7.3 Hz, 3H, CH₃), 1.29 (s, 3H, CH₃), 1.31(t, 3H, CH₃), 1.60-1.69 (m, 4H, 2CH₂), 1.97-2.08 |
| | (m, 2H), 2.15-2.31(m, 1H), 2.33-2.45 (m, 1H), 2.50-2.58 (m, 2H), 3.21 (brs, 1H), 4.20 (q, 2H, CH₂), |
| | 5.57 (s, 1H), 6.22 (s, 1H). |
| B-2 | 0.65 (d, J = 7.3 Hz, 3H, CH₃), 1.67(s, 3H, CH₃), 1.68-1.89(s, 4H, 2CH₂), 2.13-2.15 (m, 1H), 2.14 (m, |
| | 1H), 2.50-2.61 (m, 2H, CH₂), 2.91-2.94 (m, 1H), 2.99-3.01 (m, 1H), 3.18-3.19 (m, 1H), 5.85 (s, |
| | 1H), 6.49 (s, 1H), 7.08(d, 2H), 7.10(d, 2H). |
| B-3 | 0.68 (d, J = 7.3 Hz, 3H, CH₃), 1.67(s, 3H, CH₃), 1.68-1.89(s, 4H, 2CH₂), 2.13-2.15 (m, 1H), 2.14 (m, |
| | 1H), 2.50-2.61 (m, 2H, CH₂), 2.91-2.94 (m, 1H), 2.99-3.01 (m, 1H), 3.18-3.19 (m, 1H), 5.88 (s, |
| | 1H), 6.56 (s, 1H), 7.19-7.24(m, 2H), 7.30-7.34(m, 1H), 7.46-7.48(m, 1H). |
| B-4 | 0.69 (d, J = 7.3 Hz, 3H, CH₃), 1.67 (s, 3H, CH₃), 1.68-1.89 (s, 4H, 2CH₂), 2.13-2.15 (m, 1H), 2.14 |
| | (m, 1H), 2.50-2.61 (m, 2H, CH₂), 2.91-2.94 (m, 1H), 2.99-3.01 (m, 1H), 3.18-3.19 (m, 1H), 5.86 (s, |
| | 1H), 6.49 (s, 1H), 7.01-7.05 (m, 2H), 7.50-7.54 (m, 2H). |
| B-5 | 0.69 (d, J = 7.3 Hz, 3H, CH₃), 1.67(s, 3H, CH₃), 1.68-1.89(s, 4H, 2CH₂), 2.13-2.15 (m, 1H), 2.14 (m, |
| | 1H), 2.50-2.61 (m, 2H, CH₂), 2.91-2.94 (m, 1H), 2.99-3.01 (m, 1H), 3.18-3.19 (m, 1H), 5.86 (s, |
| | 1H), 6.49 (s, 1H), 7.21-7.24(m, 2H), 7.31-7.34(m, 1H), 7.45-7.48(m, 1H). |
| B-6 | 0.68 (d, J = 7.3 Hz, 3H, CH₃), 1.67(s, 3H, CH₃), 1.68-1.89(s, 4H, 2CH₂), 2.13-2.15 (m, 1H), 2.14 (m, |
| | 1H), 2.50-2.61 (m, 2H, CH₂), 2.91-2.94 (m, 1H), 2.99-3.01 (m, 1H), 3.18-3.19 (m, 1H), 5.86 (s, |
| | 1H), 6.49 (s, 1H), 7.06-7.10(m, 2H), 7.35-7.39(m, 2H). |
| B-7 | 0.68 (d, J = 7.3 Hz, 3H, CH₃), 1.67(s, 3H, CH₃), 1.68-1.89(s, 4H, 2CH₂), 2.13-2.15 (m, 1H), 2.14 (m, |
| | 1H), 2.50-2.61 (m, 2H, CH₂), 2.91-2.94 (m, 1H), 2.99-3.01 (m, 1H), 3.18-3.19 (m, 1H), 5.89 (s, |
| | 1H), 6.56 (s, 1H), 7.14-7.16(d, 2H), 7.28-7.31(m, 1H), 7.48(d, 1H). |
| B-8 | 0.62-0.64(d, J = 7.3 Hz, 3H, CH₃), 1.67(s, 3H, CH₃), 1.75-1.88(m, 4H), 2.05-2.09(d, 1H), 2.16(m, 1H), |
| | 2.51-2.64(m, 2H), 2.90-3.03(m, 2H), 3.21 (m, 1H), 5.81 (s, 1H), 6.45(s, 1H), 6.72(s, 1H), 6.84-6.86(d, 2H), |
| | 6.97-6.99(d, 2H). |
| B-9 | 0.67-0.69(d, J = 8 Hz, 3H, CH₃), 1.67(s, 3H, CH₃), 1.72-1.89(m, 4H), 2.03-2.05(m, 1H), 2.14-2.16(m, 1H), |
| | 2.38(s, 3H, CH₃), 2.51-2.64(m, 2H), 2.95-3.04(m, 2H), 3.20-3.22(m, 1H), 5.81(s, 1H), 6.48(s, 1H), |
| | 7.00-7.02(d, 2H), 7.18-7.21(d, 2H). |
| B-11 | 0.66 (d, J = 7.2 Hz, 3H, CH₃), 1.67-1.69 (s, 4H, 2CH₂), 1.87 (s, 3H, CH₃), 2.03 (m, 1H), 2.14 (m, |
| | 1H), 2.52-2.62 (m, 2H, CH₂), 2.91-2.95 (m, 1H), 3.01-3.05 (m, 1H), 3.20 (m, 1H), 5.22 (s, 2H, |
| | CH₂), 5.65 (s, 1H), 6.28 (s, 1H), 7.36-7.38 (m, 5H, Ph-H). |
| B-12 | 0.67 (d, J = 7.3 Hz, 3H, CH₃), 1.66-1.71 (m, 4H, 2CH₂), 1.87 (m, 3H, CH₃), 2.00-2.06 (m, 1H), 2.16 |
| | (m, 1H), 2.50-2.66 (m, 2H, CH₂), 2.90-2.97 (m, 1H), 3.08 (m, 1H), 3.21 (m, 1H), 5.17 (s, 2H, |
| | CH₂), 5.65 (s, 1H), 6.22 (s, 1H), 7.01-7.03 (d, J = 8 Hz, 2H), 7.35-7.37 (d, 7 = 8 Hz, 2H). |
| B-13 | 0.66 (d, J = 7.3 Hz, 3H, CH₃), 1.66-1.71 (m, 4H, 2CH₂), 1.85 (m, 3H, CH₃), 2.01-2.06 (m, 1H), 2.16 |
| | (m, 1H), 2.50-2.67 (m, 2H, CH₂), 2.90-2.97 (m, 1H), 3.11 (m, 1H), 3.19 (m, 1H), 5.27 (s, 2H, CH₂), |
| | 5.70 (s, 1H), 6.30 (s, 1H), 7.48-7.50 (d, 2H, J = 8 Hz), 7.63-7.65 (d, 2H, J = 8 Hz). |
| B-14 | 0.66 (d, J = 7.3 Hz, 3H, CH₃), 1.65-1.68 (s, 4H, 2CH₂), 1.87 (s, 3H, CH₃), 2.12-2.13 (m, 1H), 2.14 |
| | (m, 1H), 2.50-2.61 (m, 2H, CH₂), 2.91-2.94 (m, 1H), 2.99-3.01 (m, 1H), 3.18-3.19 (m, 1H), 5.16 (s, |
| | 2H, CH₂), 5.66 (s, 1H), 6.26 (s, 1H), 7.30-7.32 (d, 2H, J = 8 Hz, 2H), 7.33-7.35 (d, J = 8 Hz, 2H). |
| B-15 | 0.66(d, J = 7.2 Hz, 3H, CH₃), 1.66-1.69 (s, 4H, 2CH₂), 1.87 (s, 3H, CH₃), 2.05 (m, 1H), 2.14 (m, |
| | 1H), 2.33 (s, 3H, Ph-CH₃), 2.51-2.61 (m, 2H, CH₂), 2.91-2.94 (m, 1H), 3.00-3.04 (m, 1H), 3.19 (m, |
| | 1H), 5.31 (s, 2H, CH₂), 5.73 (s, 1H), 6.32 (s, 1H), 7.53-7.55 (d, J = 8 Hz, 2H), 8.23-8.25 (d, J = 8 |
| | Hz, 2H). |
| B-16 | 0.65 (d, J = 7.3 Hz, 3H, CH₃), 1.64-1.69 (s, 4H, 2CH₂), 1.86 (s, 3H, CH₃), 2.03-2.05 (m, 1H), 2.14 |
| | (m, 1H), 2.51-2.62 (m, 2H, CH₂), 2.91-2.94 (m, 1H), 2.99-3.01 (m, 1H), 3.19 (m, 1H), 5.16 (s, 2H, |
| | CH₂), 5.66 (s, 1H), 6.26 (s, 1H), 7.23-7.25 (d, J = 8 Hz, 2H), 7.47-7.49 (d, J = 8 Hz, 2H). |
| B-17 | 0.68 (d, J = 7.2 Hz, 3H, CH₃), 1.67 (s, 4H, 2CH₂), 1.83 (s, 3H, CH₃), 2.05 (m, 1H), 2.13 (m, 1H), |
| | 2.53-2.62 (m, 2H, CH₂), 2.90-2.94 (m, 1H), 2.99-3.01 (m, 1H), 3.19 (m, 1H), 5.26 (s, 2H, CH₂), |
| | 5.67 (s, 1H), 6.31 (s, 1H), 7.15-7.23 (m, 1H), 7.30-7.35 (m, 1H), 7.41-7.43 (m, 1H), 7.54-7.59 (m, |
| | 1H). |
| B-18 | 0.66 (d, J = 7.2 Hz, 3H, CH₃), 1.67 (s, 4H, 2CH₂), 1.83 (s, 3H, CH₃), 2.05 (m, 1H), 2.13 (m, 1H), |
| | 2.53-2.61 (m, 2H, CH₂), 2.90-2.94 (m, 1H), 2.99-3.01 (m, 1H), 3.18 (m, 1H), 3.81 (s, 3H, OCH₃), |
| | 5.15 (s, 2H, CH₂), 5.62 (s, 1H), 6.23 (s, 1H), 6.88-6.90 (d, J = 8 Hz, 2H), 7.31-7.33 (d, J = 8 Hz, 2H) |
| B-19 | 0.67 (d, J = 7.3 Hz, 3H, CH₃), 1.66-1.71 (m, 4H, 2CH₂), 1.87 (m, 3H, CH₃), 2.00-2.06 (m, 1H), |
| | 2.16 (m, 1H), 2.50-2.66 (m, 2H, CH₂), 2.90-2.97 (m, 1H), 3.08 (m, 1H), 3.21 (m, 1H), 5.27 (s, 2H, |
| | CH₂), 5.68 (s, 1H), 6.28 (s, 1H), 7.24-7.27 (d, d, J = 2, 2 Hz, 1H), 7.36-7.38 (d, J = 8 Hz, 2H), |
| | 7.421-7.426 (d, 1H, J = 2 Hz, 2H). |
| B-20 | 0.65 (d, J = 7.3 Hz, 3H, CH₃), 1.66-1.69 (s, 4H, 2CH₂), 1.88 (s, 3H, CH₃), 2.13-2.15 (m, 1H), 2.14 |
| | (m, 1H), 2.50-2.61 (m, 2H, CH₂), 2.91-2.94 (m, 1H), 2.99-3.01 (m, 1H), 3.18-3.19 (m, 1H), 5.32 (s, |
| | 2H, CH₂), 5.66 (s, 1H), 6.29 (s, 1H), 7.27-7.31 (m, 2H), 7.39-7.44 (m, 2H). |

### Example 3

Rupestonic acid was prepared according to Example 1.

### Preparation of the Inorganic Salts and Organic Salts of Rupestonic Amide derivatives (A-type compound)

### (1) Preparation of the hydrochloric salt of nitrogen (2-bromo-phenyl)-Rupestonic amide(A-5)

First, 0.5 mmol nitrogen (2-bromo-phenyl)-Rupestonic amide was added into 20 Milli-Liters of 5% hydrochloric acid solution and dissolved therein while being gently heated and stirred. A suitable amount of ethanol was added into the solution, followed by chill-crystallizing, filtering and vacuum-drying. The resultant product was the hydrochloric salt of nitrogen (2-bromo-phenyl)-Rupestonic amide, with a yield of 66% by weight.

### (2) Preparation for the acetate of nitrogen (2-bromo-phenyl)-Rupestonic amide(A-5)

First, 0.5 mmol A-5 was added into a 50 milli-Liters singly-opened round-bottom flask containing 10 mL dichloromethane. Second, 2 milli-Liters of glacial acetic acid was added and stirred at 30-40° C. for 1-2 hours. After cooling down, the solution was chill-crystallized, filtered and vacuum-dried. The resultant product was acetate of nitrogen (2-bromo-phenyl)-Rupestonic amide, with a yield of ˜58% by weight.

TABLE 6
Salts formed between A-type compounds and organic acids and inorganic acids, respectively
Salts formed between A-type compounds and organic acids or inorganic acids, respectively

| | | |
|---|---|---|
| 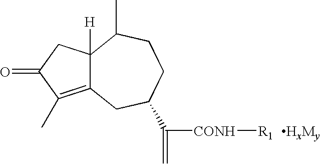 | HₓM_{y} hydrochloric acid Sulfuric acid nitric acid Phosphoric acid | inorganic salt of A-type compound hydrochloric salt of A-type compound sulfate of A-type compound nitrate of A-type compound phosphate of A-type compound |
| 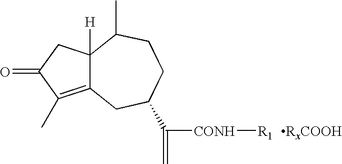 | RₓCOOH Formic acid oxalic acid acetic acid citric acid Fumaric acid Maleic acid Amino acid | organic salts of A-type compound formate of A-type compound Oxalate of A-type compound Acetate of A-type compound Citrate of A-type compound fumarate of A-type compound maleate of A-type compound amino acid salts of A-type compound |

### Example 4

### Activity Screen

The compounds shown by the formulas described above or the salts thereof were used to prepare medicines for treating anti-flu viruses or anti-herpes viruses. The bioactivity of the listed compounds was determined by the following method:

### 1. Measurement of the Anti-A, B-Type Flu Virus Activity

MDCK (Madin-darby canine kidney) cells were used as host cells for viruses. For each sample, the inhibition of the virus-induced cytopathic effect (CPE) was measured.

### Testing Materials and Method

(1) Virus sp.: in September, 2006, A-type flu virus (jifang/90-15) and B-type flu virus (jijfang/97-13) were cultured in allantoic cavities of chick embryos for passage, and the culture was stored at −80° C.

(2) Treatment of samples: samples were dissolved in DMSO, brought to a suitable initial concentration with the culture fluid, and diluted three (3) times with the culture fluid, with a dilution factor of eight (8) each.

(3) Positive control: ribovirin (RBV), manufactured by Zhejiang Kangyu Pharmaceutical Co., Ltd. (batch number 960501).

(4) Testing method: MDCK cells were inoculated in a 96-well culture plate, cultured in 5% CO₂, 37° C. for 24 hours. A-type flu viruses 10⁻³ (100×TCID₅₀) and B-type flu viruses 10⁻² (30×TCID₅₀) were added into the MDCK cells. After adsorption at 37° C. for 2 hours, the virus solution was decanted, and the drugs with various dilution factors were added respectively. The virus control and cell control were set up, cultured at 37° C. for 36 hours, and observed. CPE was recorded and anti-flu virus inhibition concentration 50% (IC₅₀) was calculated for each sample.

### 2. Measurement of the Anti-I, II Herpes Simplex Virus Activity

Vero cells were used as host cells for viruses. For samples, the inhibition of I, II herpes virus-caused cell pathological extent (CPE) was measured.

### Testing Materials and Method:

(1) Virus sp.: HSV-I, VR733 sp., HSV-II, SAV sp., both provided by ATCC.

(2) Treatment of samples: All reagents should be freshly prepared prior to use. Samples were dissolved in DMSO, brought to a suitable concentration, and diluted three (3) times with the culture fluid during the examination, with a dilution factor of eight (8) for each.

(3) Positive control: acyclovir (ACV), manufactured by Hubei Keyi Pharmaceutical Co.

(4) Testing method: Vero cells were inoculated in a 96-well culture plate. The cells were grown at 37° C. in a humidified 5% CO₂ for 24 hours. I-type herpes viruses 10⁻³ (50×TCID₅₀ infection amount) and II-type herpes viruses 10⁻⁴ (17×TCID₅₀ infection amount) are added into Vero cells respectively. After adsorption for 2 hours, the virus solution was removed, and the samples and the positive control were added according to the above dilution factors. At the same time, a cell control well and a virus control well were set up. After the observation of cell pathological extent (CPE) for 48 hours, the inhibition concentration 50% (IC₅₀) of 1-type and II-type herpes simplex viruses was calculated for each sample.

The activity of the compounds listed by the formulas of rupestonic acid derivatives described in the invention were determined by an activity testing method, and the results were shown as follows:

TABLE 6
The testing results of the anti-flu virus
activity for some preferred compounds

| | | Activity for inhibiting | | Activity for inhibiting | |
|---|---|---|---|---|---|
| | | A-type flu viruses | | B-type flu viruses | |
| Code of | TC₅₀ | IC₅₀ | | IC₅₀ | |
| compound | (μmol/L) | (μmol/L) | SI | (μmol/L) | SI |
| 1 | 1043.7 | — | — | 115.7 | 9.00 |
| A-2 | 120.6 | 19.2 | 6.3 | 29.9 | 4.0 |
| A-5 | 159.6 | 49.0 | 3.3 | — | — |
| B-1 | 193.6 | 39.1 | 4.9 | 50.9 | 3.8 |
| B-2 | 240.5 | 19.1 | 12.6 | — | — |
| B-3 | 49.7 | 11.5 | 4.3 | — | — |
| B-4 | 44.2 | 10.2 | 4.3 | — | — |
| B-9 | 191.3 | 13.4 | 14.3 | — | — |
| B-14 | 47.7 | 12.5 | 3.8 | 19.1 | 2.5 |
| B-15 | 25.0 | 5.5 | 4.6 | 5.5 | 4.6 |
| B-17 | 57.3 | 15.2 | 3.9 | 25.7 | 2.2 |
| Ribavirin | 1572.5 | 1.6 | 982.8 | 11.9 | 132.1 |
| Oseltamivir | >1219.5 | 5.1 | >239.1 | — | — |

“TC₅₀”: toxic concentration 50%,
“—”: Samples have no anti-flu virus activity under the maximum nontoxic dose.
“SI”: selection index, SI = TC₅₀/IC₅₀,
“1”: mother compound(rupestonic acid).
“RBV”: representing the positive control Ribavirin.
“Oseltamivir”: representing the positive control.

We claim:

1. A rupestonic acid derivative composition comprising: a rupestonic acid derivative compound with formula A or B or pharmaceutically acceptable salts thereof 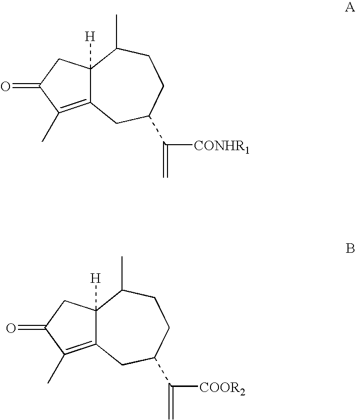
in said rupestonic acid derivative compound of formula A, R₁ is n-dodecyl, p-chlorophenyl, o-chlorophenyl, p-bromophenyl, o-bromo-phenyl, 2,4-dichlorophenyl or benzyl; and
in said rupestonic acid derivative compound of formula B, R₂ is ethyl or p-chlorophenyl, o-chlorophenyl, p-bromophenyl, o-bromophenyl, p-fluorophenyl, 2,4-dichlorophenyl, p-hydroxylphenyl, p-methylphenyl, benzyl, p-fluorobenzyl, p-trifluoromethylbenzyl, p-chlorobenzyl, p-nitrylbenzyl, p-bromobenzyl, o-bromobenzyl, p-methoxybenzyl, 2,4-dichlorobenzyl, or o-chlorobenzyl.

2. A rupestonic acid derivative according to claim 1, wherein said rupestonic acid derivative compound of formula A is any one of the following compounds:
nitrogen (n-dodecyl)-Rupestonic amide,
nitrogen (4-chloro-phenyl)-Rupestonic amide,
nitrogen (2-chloro-phenyl)-Rupestonic amide,
nitrogen (4-bromo-phenyl)-Rupestonic amide,
nitrogen (2-bromo-phenyl)-Rupestonic amide,
nitrogen (2,4-dichloro-phenyl)-Rupestonic amide, or
nitrogen (benzyl)-Rupestonic amide.

3. A rupestonic acid derivative according to claim 1, wherein said rupestonic acid derivative compound of formula B is any one of the following compounds:
Ethyl Rupestonic ester,
Rupestonic (p-chlorophenyl)-ester,
Rupestonic (o-chlorophenyl)-ester,
Rupestonic (p-bromophenyl)-ester,
Rupestonic (o-bromophenyl)-ester,
Rupestonic (p-fluorophenyl)-ester,
Rupestonic (2,4-dichloro-phenyl)-ester,
Rupestonic (p-hydroxyl-phenyl)-ester,
Rupestonic (p-methylphenyl)-ester,
Rupestonic (p-cyano-phenyl)-ester,
Benzyl Rupestonic ester,
Rupestonic (p-fluorobenzyl)-ester,
Rupestonic (p-trifluoromethyl-benzyl)-ester,
Rupestonic (p-chlorobenzyl)-ester,
Rupestonic (p-nitryl-benzyl)-ester,
Rupestonic (p-bromobenzyl)-ester,
Rupestonic (o-bromobenzyl)-ester,
Rupestonic (p-methoxy-benzyl)-ester,
Rupestonic (2,4-dichloro-benzyl)-ester,
Rupestonic (o-chlorobenzyl)-ester, or
Rupestonic (p-cyano-benzyl)-ester.

4. A rupestonic acid derivative according to claim 1, wherein said pharmaceutically acceptable salts are formed from said rupestonic acid derivative compounds of formula A and include hydrochloric salts, sulphate salts, phosphate salts, nitrate salts, formate salts, oxalate salts, acetate salts, citrate salts, fumarate salts, maleate salts, amino acid salts, or a combination thereof.

5. A rupestonic acid derivative according to claim 1, wherein any one of said compounds, as an active ingredient, constitutes a pharmaceutical composition either alone or in combination with one or more pharmaceutically acceptable, inert and nontoxic excipients or carriers.

6. A process for preparing a rupestonic acid derivative of formula A 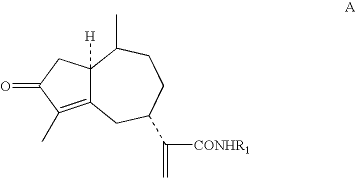
R₁ is n-dodecyl, p-chlorophenyl, o-chlorophenyl, p-bromophenyl, o-bromo-phenyl, 2,4-dichlorophenyl or benzyl,
comprised of reacting a rupestonic acid monomer compound and an amine compound through an acylamide reaction.

7. The process of claim 6, wherein the rupestonic acid monomer is rupestonic acid and the amine compound is selected from one of an aromatic amine or fatty amine.

8. A process for preparing a rupestonic acid derivative of formula B 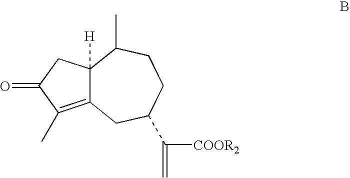
R₂ is ethyl or p-chlorophenyl, o-chlorophenyl, p-bromophenyl, o-bromophenyl, p-fluorophenyl, 2,4-dichlorophenyl, p-hydroxylphenyl, p-methylphenyl, benzyl, p-fluorobenzyl, p-trifluoromethylbenzyl, p-chlorobenzyl, p-nitrylbenzyl, p-bromobenzyl, o-bromobenzyl, p-methoxybenzyl, 2,4-dichlorobenzyl, or o-chlorobenzyl, comprising: reacting a rupestonic acid monomer compound and an alcohol compound through an esterification reaction.

9. The process of claim 8, wherein the Ruestonic acid monomer is rupestonic acid and the alcohol compound is selected from one of an aromatic alcohol or a fatty alcohol.

10. A method of treating flu virus, which comprises administering to a patient in need thereof a pharmaceutically effective amount of a composition comprised of a rupestonic acid derivative compound of a selected on of formula A or Formula B, 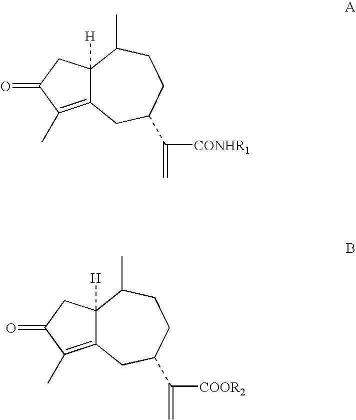
in said rupestonic acid derivative compound of formula A, R₁ is n-dodecyl, p-chlorophenyl, o-chlorophenyl, p-bromophenyl, o-bromo-phenyl, 2,4-dichlorophenyl or benzyl;
in said rupestonic acid derivative compound of formula B, R₂ is ethyl or p-chlorophenyl, o-chlorophenyl, p-bromophenyl, o-bromophenyl, p-fluorophenyl, 2,4-dichlorophenyl, p-hydroxylphenyl, p-methylphenyl, benzyl, p-fluorobenzyl, p-trifluoromethylbenzyl, p-chlorobenzyl, p-nitrylbenzyl, p-bromobenzyl, o-bromobenzyl, p-methoxybenzyl, 2,4-dichlorobenzyl, or o-chlorobenzyl.

11. The method of claim 10, wherein the flu virus is A-type flu virus.

12. The method of claim 10, wherein the flu virus is B-type flu virus.

13. A method of treating herpes virus comprising administering to a patient in need thereof a pharmaceutically effective amount of a composition comprises a rupestonic acid derivative compound of a selected one of formula A or formula B, 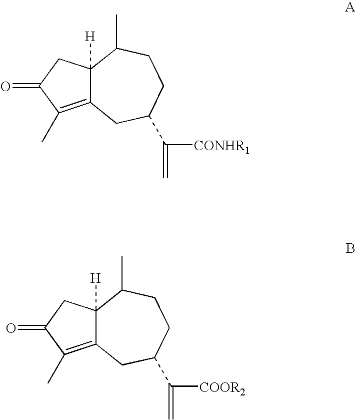
in said rupestonic acid derivative compound of formula A, R₁ is n-dodecyl, p-chlorophenyl, o-chlorophenyl, p-bromophenyl, o-bromo-phenyl, 2,4-dichlorophenyl or benzyl;
in said rupestonic acid derivative compound of formula B, R₂ is ethyl or p-chlorophenyl, o-chlorophenyl, p-bromophenyl, o-bromophenyl, p-fluorophenyl, 2,4-dichlorophenyl, p-hydroxylphenyl, p-methylphenyl, benzyl, p-fluorobenzyl, p-trifluoromethylbenzyl, p-chlorobenzyl, p-nitrylbenzyl, p-bromobenzyl, o-bromobenzyl, p-methoxybenzyl, 2,4-dichlorobenzyl, or o-chlorobenzyl.

14. The method of claim 13, wherein the herpes virus is I-type herpes virus.

15. The method of claim 13, wherein the herpes virus is II-type herpes virus.

16. The method of claim 10, wherein the rupestonic acid derivative is of formula A and is any one of the following compounds:
nitrogen (n-dodecyl)-Rupestonic amide,
nitrogen (4-chloro-phenyl)-Rupestonic amide,
nitrogen (2-chloro-phenyl)-Rupestonic amide,
nitrogen (4-bromo-phenyl)-Rupestonic amide,
nitrogen (2-bromo-phenyl)-Rupestonic amide,
nitrogen (2,4-dichloro-phenyl)-Rupestonic amide, or
nitrogen (benzyl)-Rupestonic amide.

17. The method of claim 10, wherein the rupestonic acid derivative is of formula B and is any one of the following compounds:
Ethyl Rupestonic ester,
Rupestonic (p-chlorophenyl)-ester,
Rupestonic (o-chlorophenyl)-ester,
Rupestonic (p-bromophenyl)-ester,
Rupestonic (o-bromophenyl)-ester,
Rupestonic (p-fluorophenyl)-ester,
Rupestonic (2,4-dichloro-phenyl)-ester,
Rupestonic (p-hydroxyl-phenyl)-ester,
Rupestonic (p-methylphenyl)-ester,
Rupestonic (p-cyano-phenyl)-ester,
Benzyl Rupestonic ester,
Rupestonic (p-fluorobenzyl)-ester,
Rupestonic (p-trifluoromethyl-benzyl)-ester,
Rupestonic (p-chlorobenzyl)-ester,
Rupestonic (p-nitryl-benzyl)-ester,
Rupestonic (p-bromobenzyl)-ester,
Rupestonic (o-bromobenzyl)-ester,
Rupestonic (p-methoxy-benzyl)-ester,
Rupestonic (2,4-dichloro-benzyl)-ester,
Rupestonic (o-chlorobenzyl)-ester, or
Rupestonic (p-cyano-benzyl)-ester.

18. The method of claim 13, wherein the rupestonic acid derivative is of formula A and is any one of the following compounds:
nitrogen (n-dodecyl)-Rupestonic amide,
nitrogen (4-chloro-phenyl)-Rupestonic amide,
nitrogen (2-chloro-phenyl)-Rupestonic amide,
nitrogen (4-bromo-phenyl)-Rupestonic amide,
nitrogen (2-bromo-phenyl)-Rupestonic amide,
nitrogen (2,4-dichloro-phenyl)-Rupestonic amide, or
nitrogen (benzyl)-Rupestonic amide.

19. The method of claim 13, wherein the rupestonic acid derivative is of formula B and is any one of the following compounds:
Ethyl Rupestonic ester,
Rupestonic (p-chlorophenyl)-ester,
Rupestonic (o-chlorophenyl)-ester,
Rupestonic (p-bromophenyl)-ester,
Rupestonic (o-bromophenyl)-ester,
Rupestonic (p-fluorophenyl)-ester,
Rupestonic (2,4-dichloro-phenyl)-ester,
Rupestonic (p-hydroxyl-phenyl)-ester,
Rupestonic (p-methylphenyl)-ester,
Rupestonic (p-cyano-phenyl)-ester,
Benzyl Rupestonic ester,
Rupestonic (p-fluorobenzyl)-ester,
Rupestonic (p-trifluoromethyl-benzyl)-ester,
Rupestonic (p-chlorobenzyl)-ester,
Rupestonic (p-nitryl-benzyl)-ester,
Rupestonic (p-bromobenzyl)-ester,
Rupestonic (o-bromobenzyl)-ester,
Rupestonic (p-methoxy-benzyl)-ester,
Rupestonic (2,4-dichloro-benzyl)-ester,
Rupestonic (o-chlorobenzyl)-ester, or
Rupestonic (p-cyano-benzyl)-ester.
